(19) United States
(12) Patent Application Publication
(10) Pub. No.: US 2004/0131563 A1
(43) Pub. Date: Jul. 8, 2004

(54) Use of active substance combinations from alpha lipoic acid and substances that absorb light in the uv-a and/or uv-b range for use in the treatment and/or prophylaxis of undesired cutaneous pigmentation

(76) Inventors: Claudia Mundt, Bremen (DE); Jens Schulz, Berlin (DE); Uwe Schonrock, Nahe (DE); Rainer Wolber, Hamburg (DE); Inge Kruse, Hamburg (DE)

Correspondence Address: GREENBLUM & BERNSTEIN, P.L.C., 1950 ROLAND CLARKE PLACE, RESTON, VA, 20191, US

(21) Appl. No.: 10/469,240

(22) PCT Filed: Mar. 5, 2002
(86) PCT No.: PCT/EP02/02373

(30) Foreign Application Priority Data
Mar. 6, 2001 (DE) 101 11 046.4

(51) Int. Cl. A61K 7/42; A61K 31/385

(52) U.S. Cl. 424/59; 514/440

(57) ABSTRACT

Use of active ingredient combinations of

(a) α-lipoic acid and

(b) one or more dermatologically compatible substances which bring about light absorption in the UV-A region and/or UV-B region,

for the preparation of cosmetic or dermatological preparations for the preparation of cosmetic or dermatological preparations for the treatment and/or prophylaxis of pigmentation disorders.

The present invention relates to the use of active ingredient combinations of α-lipoic acid and dermatologically compatible substances which bring about light absorption in the UV-A region and/or UV-B region for the preparation of cosmetic or dermatological preparations for the prophylaxis and treatment of cosmetic or dermatological skin conditions, such as, for example, undesired pigmentation, for example local hyperpigmentation and incorrect pigmentation (for example liver spots, freckles), or for the purely cosmetic lightening of relatively large areas of skin which are quite appropriately pigmented for the individual skin type.

Melanocytes are responsible for the pigmenting of the skin; these are found in the lowest layer of the epidermis, the Stratum basale, alongside the basal cells as pigment-forming cells which, depending on skin type, occur either individually or in clusters of varying size. Melanocytes contain, as characteristic cell organelles, melanosomes which form melanin to an increased degree when stimulated by UV radiation. This melanin is transported to the keratinocytes and brings about a more or less marked brownish or brown skin color.

Melanin is formed as the end stage of an oxidation process in which tyrosine is ultimately converted into melanin, under the co-action of the enzyme tyrosinase, via 3,4-dihydroxyphenylalanine (dopa), dopaquinone, leucodopachrome, dopachrome, 5,6-dihydroxyindole and indole-5,6-quinone.

Problems with skin hyperpigmentation have many different causes and are accompanying phenomena of many biological processes, for example UV radiation (for example freckles, Ephelides), genetic disposition, incorrect pigmentation of the skin during wound healing or scarring or skin aging (e.g. Lentigines seniles).

Active ingredients and preparations which counteract skin pigmentation are known. In practice, use is essentially made of preparations based on hydroquinone although, on the one hand, these only exhibit their effect after they have been applied for several weeks and, on the other hand, application of them for an excessively long time is not without risk for toxicological reasons. The inhibition of tyrosinase with substances such as kojic acid, ascorbic acid and azelaic acid, and derivatives thereof, is common, but has cosmetic and dermatological disadvantages.

The object of the present invention was to remedy these shortcomings.

According to the invention, the shortcomings of the prior art are eliminated through the use of active ingredient combinations of

(a) α-lipoic acid and

(b) one or more dermatologically compatible substances which bring about light absorption in the UV-A region and/or UV-B region,

for the preparation of cosmetic or dermatological preparations for the preparation of cosmetic or dermatological preparations for the treatment and/or prophylaxis of pigmentation disorders.

The active ingredient combinations according to the invention and cosmetic or dermatological preparations comprising such active ingredient combinations are entirely satisfactory preparations in every respect. It could not have been foreseen by the person skilled in the art that the preparations according to the invention are more effective against pigment disorders than the preparations of the prior art.

The use of the active ingredient combinations used according to the invention or of cosmetic or topical dermatological preparations with an effective content of active ingredient combinations used according to the invention surprisingly enables effective treatment, but also prophylaxis of pigmentation disorders.

The active ingredient combinations according to the invention have a synergistic effect with regard to the individual components, in all of these uses.

α-Lipoic acid was isolated in 1952 from liver tissue and its structure was explained as a sulfur-containing fatty acid. Bacteria, plants and higher organisms can produce α-lipoic acid themselves in their metabolism; the question of whether humans biosynthesize their own α-lipoic acid is still open.

α-Lipoic acid is used in the therapy of polyneuropathy, a sensibility disorder in the hands and feet as a long-term effect of diabetes. 200 to 600 milligrams of α-lipoic acid per day lead to a significant reduction in pain intensity. The energy metabolism of the nerves in the hands and feet is activated by α-lipoic acid, resulting in better nerve conductivity and thus fewer feelings of numbness and reflex losses.

α-Lipoic acid reduces pathologically increased liver function values and promotes the healing of hepatitis. α-Lipoic acid is present in most foods in small amounts, relatively high contents only being found in meat. It is recognized that α-lipoic acid has strongly antioxidative properties.

WO 97/10808 and U.S. Pat. No. 5,472,698 describe the cosmetic use of α-lipoic acid against symptoms of skin aging. DE-42 42 876 describes active ingredient combinations of biotin and antioxidants with α-lipoic acid for the cosmetic and/or dermatological care of the skin and/or skin appendages, and also cosmetic and/or dermatological preparations comprising such active ingredient combinations.

Advantageously, the preparations according to the invention comprise 0.001-10% by weight of α-lipoic acid, based on the total weight of the preparations.

The preparations according to the invention comprise substances which absorb UV radiation in the UV-A and/or UV-B region, where the total amount of filter substances is, for example, 0.1% by weight to 30% by weight, preferably 0.5 to 20% by weight, in particular 1.0 to 15.0% by weight, based on the total weight of the preparations, in order to make available cosmetic preparations which protect the hair or the skin from the entire range of ultraviolet radiation. They can also be used as sunscreen compositions for the hair or the skin.

Advantageous UV-A filter substances for the purposes of the present invention are dibenzoylmethane derivatives, in particular 4-(tert-butyl)-4′-methoxydibenzoylmethane (CAS No. 70356-09-1), which is sold by Givaudan under the name Parsol® 1789 and by Merck under the trade name Eusolex®) 9020.

Further advantageous UV-A filter substances are phenylene-1,4-bis(2-benzimidazyl)-3,3′-5,5′-tetrasulfonic acid

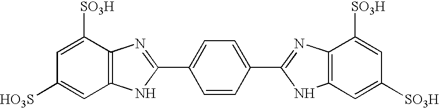

and its salts, particularly the corresponding sodium, potassium or triethanolammonium salts, in particular the phenylene-1,4-bis(2-benzimidazyl)-3,3′-5,5′-tetrasulfonic acid bis-sodium salt

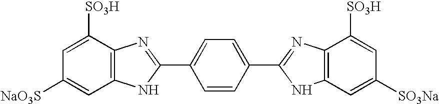

having the INCl name Bisimidazylate, which is obtainable, for example, under the trade name Neo Heliopan AP from Haarmann & Reimer.

Also advantageous are 1,4-di(2-oxo-10-sulfo-3-bornylidenemethyl)benzene and salts thereof (particularly the corresponding 10-sulfato compounds, in particular the corresponding sodium, potassium or triethanolammonium salt), which is also referred to as benzene-1,4-di(2-oxo-3-bornylidenemethyl-10-sulfonic acid) and is characterized by the following structure:

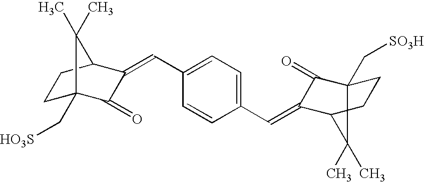

Advantageous UV filter substances for the purposes of the present invention are also broadband filters, i.e. filter substances which absorb both UV-A and also UV-B radiation.

Advantageous broadband filters or UV-B filter substances are, for example, bis-resorcinyltriazine derivatives having the following structure:

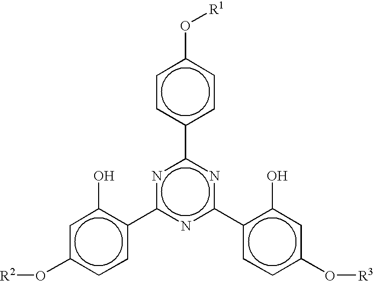

where R¹, R² and R³, independently of one another, are chosen from the group of branched and unbranched alkyl groups having 1 to 10 carbon atoms or represent a single hydrogen atom. Particular preference is given to 2,4-bis{[4-(2-ethylhexyloxy)-2-hydroxy]phenyl}-6-(4-methoxyphenyl)-1,3,5-triazine (INCl: Aniso Triazine), which is obtainable under the trade name Tinosorb® S from CIBA-Chemikalien GmbH, and the tris(2-ethylhexyl) 4,4′,4″-(1,3,5-triazine-2,4,6-triyltriimino)trisbenzoate, synonym: 2,4,6-tris[anilino(p-carbo-2′-ethyl-1′-hexyloxy)]-1,3,5-triazine (INCl: Octyl Triazone), which is sold by BASF Aktiengesellschaft under the trade name UVINUL® T 150.

Other UV filter substances which have the structural formula

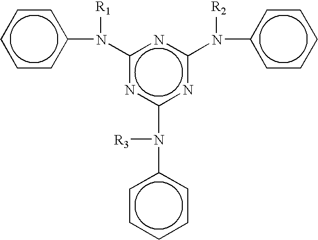

are also advantageous UV filter substances for the purposes of the present invention, for example the s-triazine derivatives described in European laid-open specification EP 570 838 A1, the chemical structure of which is given by the generic formula

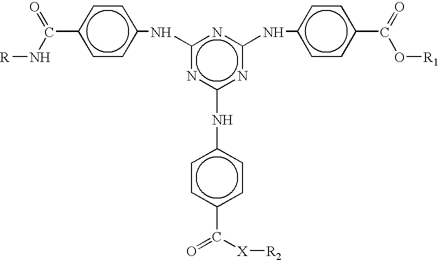

where

R is a branched or unbranched C₁-C₁₈-alkyl radical, a C₅-C₁₂-cycloalkyl radical, optionally substituted by one or more C₁-C₄-alkyl groups,

X is an oxygen atom or an NH group,

R₁ is a branched or unbranched C₁-C₁₈-alkyl radical, a C₅-C₁₂-cycloalkyl radical, optionally substituted by one or more C₁-C₄-alkyl groups, or a hydrogen atom, an alkali metal atom, an ammonium group or a group of the formula

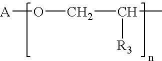

in which

A is a branched or unbranched C₁-C₁₈-alkyl radical, a C₅-C₁₂-cycloalkyl or aryl radical, optionally substituted by one or more C₁-C₄-alkyl groups,

R₃ is a hydrogen atom or a methyl group,

n is a number from 1 to 10,

R₂ is a branched or unbranched C₁-C₁₈-alkyl radical, a C₅-C₁₂-cycloalkyl radical, optionally substituted by one or more C₁-C₄-alkyl groups, if X is the NH group, and a branched or unbranched C₁-C₁₈-alkyl radical, a C₅-C₁₂-cycloalkyl radical, optionally substituted by one or more C₁-C₄-alkyl groups, or a hydrogen atom, an alkali metal atom, an ammonium group or a group of the formula

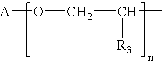

in which

A is a branched or unbranched C₁-C₁₈-alkyl radical, a C₅-C₁₂-cycloalkyl or aryl radical, optionally substituted by one or more C₁-C₄-alkyl groups,

R₃ is a hydrogen atom or a methyl group,

n is a number from 1 to 10,

if X is an oxygen atom.

A particularly advantageous UV filter substance for the purposes of the present invention is also an asymmetrically substituted s-triazine, the chemical structure of which is given by the formula

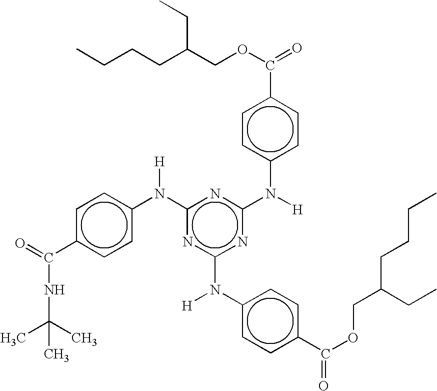

which is also referred to below as dioctylbutylamidotriazone (INCl: Dioctylbutamido-triazone) and is obtainable under the trade name UVASORB HEB from Sigma 3V.

European laid-open specification 775 698 also describes advantageous bis-resorcinyl-triazine derivatives, the chemical structure of which is given by the generic formula

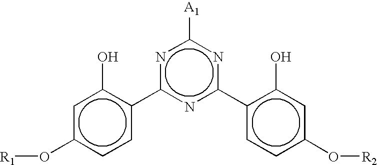

where R₁, R₂ and A₁ represent very different organic radicals.

Also advantageous for the purposes of the present invention are 2,4-bis{[4-(3-sulfonato)-2-hydroxypropyloxy)-2-hydroxylphenyl}-6-(4-methoxyphenyl)-1,3,5-triazine sodium salt, 1,3,5-triazine, 2,4-bis{[4-(2-ethylhexyloxy)-2-hydroxy]phenyl}-6-4-(2-methoxyethylcarboxyl)phenylamino]-1,3,5-triazine, 2,4-bis{[4-(3-(2-propyloxy)-2-hydroxypropyloxy)-2-hydroxy]phenyl}-6-[4-(2-ethylcarboxyl)phenylamino]-1,3,5-triazine, 2,4-bis{[4-(2-ethylhexyloxy)-2-hydroxy]phenyl}-6-(1-methylpyrrol-2-yl)-1,3,5-triazine, 2,4-bis{[4-tris(trimethylsiloxysilylpropyloxy)-2-hydroxy]phenyl}-6-(4-methoxyphenyl)-1,3,5-triazine, 2,4-bis{[4-(2″-methylpropenyloxy)-2-hydroxy]phenyl}-6-(4-methoxyphenyl)-1,3,5-triazine and 2,4-bis{[4-(1′,1′,1′,3′, 5′,5′,5′-heptamethylsiloxy-2″-methylpropyloxy)-2-hydroxy]phenyl}-6-(4-methoxyphenyl)-1,3,5-triazine.

An advantageous broadband filter for the purposes of the present invention is 2,2′-methylenebis(6-(2H-benzotriazol-2-yl)-4-(1,1,3,3-tetramethylbutyl)phenol) [I NCl: bisoctyltriazole], which is characterized by the chemical structural formula

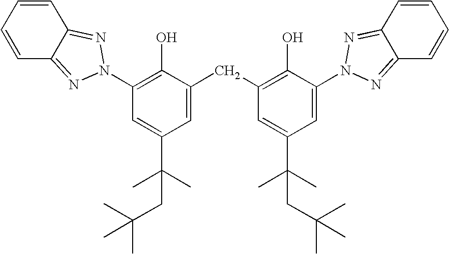

and is obtainable under the trade name Tinosorb® M from CIBA-Chemikalien GmbH.

An advantageous broadband filter for the purposes of the present invention is also 2-(2H-benzotriazol-2-yl)-4-methyl-6-[2-methyl-3-[1,3,3,3-tetramethyl-1-[(trimethylsilyl)-oxy]disiloxanyl]propyl]phenol (CAS No.: 155633-54-8) with the INCl name Drometrizole Trisiloxane, which is characterized by the chemical structural formula.

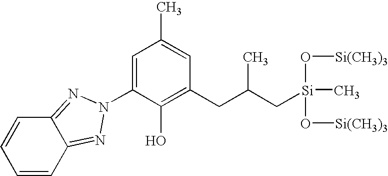

The UV-B filters may be oil-soluble or water-soluble. Advantageous oil-soluble UV-B filter substances are, for example:

3-benzylidenecamphor derivatives, preferably 3-(4-methylbenzylidene)camphor, 3-benzylidenecamphor;

4-aminobenzoic acid derivatives, preferably 2-ethylhexyl 4-(dimethylamino)benzoate, amyl 4-(dimethylamino)benzoate;

2,4,6-trianilino(p-carbo-2′-ethyl-1′-hexyloxy)-1,3,5-triazine;

esters of benzalmalonic acid, preferably di(2-ethylhexyl) 4-methoxybenzalmalonate;

esters of cinnamic acid, preferably 2-ethylhexyl 4-methoxycinnamate, isopentyl 4-methoxycinnamate;

derivatives of benzophenone, preferably 2-hydroxy-4-methoxybenzophenone, 2-hydroxy-4-methoxy-4′-methylbenzophenone, 2,2′-dihydroxy-4-methoxybenzo-phenone

and UV filters bonded to polymers.

Advantageous water-soluble UV-B filter substances are, for example:

salts of 2-phenylbenzimidazole-5-sulfonic acid, such as its sodium, potassium or its triethanolammonium salt, and the sulfonic acid itself;

sulfonic acid derivatives of 3-benzylidenecamphor, such as, for example, 4-(2-oxo-3-bornylidenemethyl)benzenesulfonic acid, 2-methyl-5-(2-oxo-3-bornylidenemethyl)-sulfonic acid and salts thereof.

A further light protection filter substance which can be used advantageously according to the invention is ethylhexyl 2-cyano-3,3-diphenylacrylate (octocrylene), which is obtainable from BASF under the name Uvinul® N 539 and is characterized by the following structure:

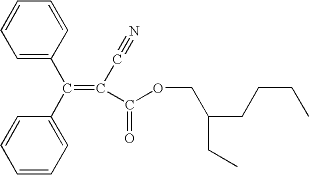

It may also be considerably advantageous to use polymer-bound or polymeric UV filter substances in preparations according to the present invention, in particular those described in WO-A-92/20690.

In addition, in some instances it may be advantageous to incorporate further UV-A and/or UV-B filters into cosmetic or dermatological preparations according to the invention, for example certain salicylic acid derivatives, such as 4-isopropylbenzyl salicylate, 2-ethylhexyl salicylate (=Octyl salicylate), homomenthyl salicylate.

The list of UV filters mentioned which can be used for the purposes of the present invention is not of course intended to be limiting.

It is advantageous according to the invention to choose the molar ratio of the substances given under (a) and (b) from the range from 10:1 to 1:10, preferably 5:1 to 1:5, particularly preferably 2:1 to 1:2.

According to the invention, it is, in particular, extremely advantageous to use the active ingredient combination used according to the invention or cosmetic or topical dermatological preparations with an active content of active ingredient combination used according to the invention for the cosmetic or dermatological treatment or prophylaxis of undesired skin conditions.

According to the invention, customary antioxidants may be used preparations which comprise the active ingredient combinations according to the invention.

The antioxidants are advantageously chosen from the group consisting of amino acids (e.g. glycine, histidine, tyrosine, tryptophan) and derivatives thereof, imidazoles (e.g. urocanic acid) and derivatives thereof, peptides, such as D,L-carnosine, D-carnosine, L-carnosine and derivatives thereof (e.g. anserine), carotenoides, carotenes (e.g. α-carotene, β-carotene, lycopene) and derivatives thereof, aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts) and sulfoximine compounds (e.g. buthionine sulfoximines, homocysteine sulfoximine, buthionine sulfones, penta-, hexa-, heptathionine sulfoximine) in very low tolerated doses (e.g. pmol to pmol/kg), and also (metal) chelating agents (e.g. α-hydroxy fatty acids, palmitic acid, phytic acid, lactoferrin) α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, alaninediacetic acid, flavonoids, polyphenols, catechins, vitamin C and derivatives (e.g. ascorbyl palmitate, Mg ascorbyl phosphate, ascorbyl acetate) tocopherols and derivatives (e.g. vitamin E acetate), and coniferyl benzoate of benzoin resin, rutinic acid and derivatives thereof, ferulic acid and derivatives thereof, butylhydroxytoluene, butylhydroxyanisol, nordihydroguaiacic acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, trans-stilbene oxide) and the derivatives (salts, esters, ethers, sugars, nucleotides, nucleosides, peptides and lipids) of these said active ingredients which are suitable according to the invention.

The amount of antioxidants (one or more compounds) in the preparations is preferably 0.001 to 30% by weight, particularly preferably 0.05-20% by weight, in particular 1-10% by weight, based on the total weight of the preparation.

The prophylaxis or the cosmetic or dermatological treatment with the active ingredient used according to the invention or with the cosmetic or topical dermatological preparations with an active content of active ingredient used according to the invention is carried out in the usual manner, by applying the active ingredient used according to the invention or the cosmetic or topical dermatological preparations with an active content of active ingredient used according to the invention to the affected areas of skin.

The active ingredient used according to the invention can advantageously be incorporated into customary cosmetic and dermatological preparations, which may be in various forms. Thus, they may, for example, be a solution, an emulsion of the water-in-oil (W/O) type or of the oil-in-water (O/W) type, or a multiple emulsions, for example of the water-in-oil-in-water (W/O/W) type or oil-in-water-in-oil (O/W/O) type, a hydrodispersion or lipodispersion, a gel, a solid stick or an aerosol.

Emulsions according to the invention for the puposes of the present invention, e.g. in the form of a cream, a lotion, a cosmetic milk, are advantageous and comprise, for example, fats, oils, waxes and/or other fatty substances, and water and one or more emulsifiers as are customarily used for this type of formulation.

It is also possible and advantageous for the purposes of the present invention to incorporate the active ingredient used according to the invention into aqueous systems or surfactant preparations for cleansing the skin and the hair.

The person skilled in the art is of course aware that demanding cosmetic compositions are mostly inconceivable without the customary auxiliaries and additives. The cosmetic preparations according to the invention can therefore comprise cosmetic auxiliaries, as are customarily used in such preparations, e.g. preservatives, bactericides, deodorizing substances, antiperspirants, insect repellents, vitamins, antifoams, dyes, pigments with a coloring action, thickeners, softening substances, moisturizing substances and/or humectant substances, fats, oils, waxes or other customary constituents of a cosmetic formulation, such as alcohols, polyols, polymers, foam stabilizers, electrolytes, organic solvents or silicone derivatives.

Corresponding requirements apply mutatis mutandis to the formulation of medicinal preparations.

Medicinal topical compositions for the purposes of the present invention generally comprise one or more medicaments in an effective concentration. For the sake of simplicity, for a clear distinction between cosmetic and medicinal application and corresponding products, reference is made to the legal provisions of the Federal Republic of Germany (e.g. Cosmetics Directive, Foods and Drugs Act).

Cosmetic and dermatological preparations according to the invention advantageously also comprise inorganic pigments based on metal oxides and/or other metal compounds which are insoluble or sparingly soluble in water, in particular the oxides of titanium (TiO₂), zinc (ZnO), iron (e.g. Fe₂O₃), zirconium (ZrO₂), silicon (SiO₂), manganese (e.g. MnO), aluminum (Al₂O₃), cerium (e.g. Ce₂O₃), mixed oxides of the corresponding metals, and mixtures of such oxides. The pigments are particularly preferably based on TiO₂.

For the purposes of the present invention, it is particularly advantageous, although not obligatory, for the inorganic pigments to be present in hydrophobic form, i.e. have been treated on the surface to repel water. This surface-treatment may involve providing the pigments with a thin hydrophobic layer by processes known per se.

One such process involves, for example, producing the hydrophobic surface layer in accordance with a reaction according to

nTiO₂+m(RO)₃Si—R′->nTiO₂ (surf.)

Here, n and m are stoichiometric parameters to be used as desired, R and R′ are the desired organic radicals. For example, hydrophobicized pigments prepared analogously to DE-A 33 14 742 are advantageous.

Advantageous TiO₂ pigments are available, for example, under the trade names MT 100 T from TAYCA, and also M 160 from Kemira and T 805 from Degussa.

Preparations according to the invention may, especially when crystalline or microcrystalline solid bodies, for example inorganic micropigments, are to be incorporated into the preparations according to the invention, also comprise anionic, nonionic and/or amphoteric surfactants. Surfactants are amphiphilic substances which can dissolve organic, nonpolar substances in water.

The hydrophilic moieties of a surfactant molecule are mostly polar functional groups, for example —COO⁻, —OSO₃²⁻, SO₃²⁻, whereas the hydrophobic moieties are usually nonpolar hydrocarbon radicals. Surfactants are generally classified according to the type and charge of the hydrophilic molecular moiety. In this connection, it is possible to differentiate between four groups:

anionic surfactants,

cationic surfactants,

amphoteric surfactants and

nonionic surfactants.

Anionic surfactants usually have, as functional groups, carboxylate, sulfate or sulfonate groups. In aqueous solution, they form negatively charged organic ions in acidic or neutral medium. Cationic surfactants are characterized almost exclusively by the presence of a quaternary ammonium group. In aqueous solution, they form positively charged organic ions in acidic or neutral medium. Amphoteric surfactants contain both anionic and cationic groups and accordingly in aqueous solution exhibit the behavior of anionic or cationic surfactants depending on the pH. In strongly acidic medium, they have a positive charge, and in alkali medium a negative charge. By contrast, in the neutral pH range, they are zwitterionic, as the example below is intended to illustrate:

| | | |
|---|---|---|
| RNH₂⁺CH₂CH₂COOH X⁻ | (at pH = 2) | X⁻ = any anion, e.g. Cl⁻ |
| RNH₂⁺CH₂CH₂COO⁻ | (at pH = 7) | |
| RNHCH₂CH₂COO⁻ B⁺ | (at pH = 12) | B⁺ = any cation, e.g. Na⁺ |

Typical nonionic surfactants are polyether chains. Nonionic surfactants do not form ions in aqueous medium.

A. Anionic Surfactants.

Anionic surfactants which can be used advantageously are acylamino acids (and salts thereof), such as

1. acyl glutamates, for example sodium acyl glutamate, di-TEA-palmitoyl aspartate and sodium caprylic/capric glutamate,

2. acylpeptides, for example palmitoyl-hydrolyzed milk protein, sodium cocoyl-hydrolyzed soya protein and sodium/potassium cocoyl-hydrolyzed collagen,

3. sarcosinates, for example myristoyl sarcosine, TEA-lauroyl sarcosinate, sodium lauroyl sarcosinate and sodium cocoyl sarcosinate,

4. taurates, for example sodium lauroyl taurate and sodium methyl cocoyl taurate,

5. acyl lactylates, lauroyl lactylate, caproyl lactylate

6. alaninates

carboxylic acids and derivatives, such as

1. carboxylic acids, for example lauric acid, aluminum stearate, magnesium alkanolate and zinc undecylenate,

2. ester carboxylic acids, for example calcium stearoyl lactylate, laureth-6 citrate and sodium PEG-4 lauramide carboxylate,

3. ether carboxylic acids, for example sodium laureth-13 carboxylate and sodium PEG-6 cocamide carboxylate,

phosphoric esters and salts, such as, for example, DEA-oleth-10 phosphate and dilaureth-4 phosphate,

sulfonic acids and salts, such as

1. acyl isethionates, e.g. sodium/ammonium cocoyl isethionate,

2. alkylarylsulfonates,

3. alkylsulfonates, for example sodium cocomonoglyceride sulfate, sodium C₁₂₋₁₄-olefinsulfonate, sodium lauryl sulfoacetate and magnesium PEG-3 cocamide sulfate,

4. sulfosuccinates, for example dioctyl sodium sulfosuccinate, disodium laureth sulfosuccinate, disodium lauryl sulfosuccinate and disodium undecylene-amido-MEA sulfosuccinate and

sulfuric esters, such as

1. alkyl ether sulfate, for example sodium, ammonium, magnesium, MIPA, TIPA laureth sulfate, sodium myreth sulfate and sodium C₁₂₋₁₃ parethsulfate,

2. alkyl sulfates, for example sodium, ammonium and TEA lauryl sulfate.

B. Cationic Surfactants

Cationic surfactants which can be used advantageously are

1. alkylamines,

2. alkylimidazoles,

3. ethoxylated amines and

4. quaternary surfactants

5. ester quats

Quaternary surfactants comprise at least one N atom which is covalently bonded to 4 alkyl and/or aryl groups. Irrespective of the pH, this leads to a positive charge.

Alkylbetaine, alkylamidopropylbetaine and alkylamidopropylhydroxysulfain are advantageous quaternary surfactants. The cationic surfactants used according to the invention can also be preferably chosen from the group of quaternary ammonium compounds, in particular benzyltrialkylammonium chlorides or bromides, such as, for example, benzyldimethylstearylammonium chloride, and also alkyltrialkylammonium salts, for example for example cetyltrimethylammonium chloride or bromide, alkyldimethylhydroxyethylammonium chlorides or bromides, dialkyldimethylammonium chlorides or bromides, alkylamidoethyltrimethylammonium ether sulfates, alkylpyridinium salts, for example lauryl- or cetylpyrimidinium chloride, imidazoline derivatives and compounds with a cationic character, such as amine oxides, for example alkyl dimethylamine oxides or alkylaminoethyldimethylamine oxides. In particular, the use of cetyltrimethylammonium salts is advantageous.

C. Amphoteric Surfactants

Amphoteric surfactants which can be used advantageously are

1. acyl/dialkylethylenediamine, for example sodium acyl amphoacetate, disodium acyl amphodipropionate, disodium alkyl amphodiacetate, sodium acyl amphohydroxypropylsulfonate, disodium acyl amphodiacetate and sodium acyl amphopropionate,

2. N-alkylamino acids, for example aminopropylalkylglutamide, alkylaminopropionic acid, sodium alkylimidodipropionate and lauroamphocarboxyglycinate.

D. Nonionic Surfactants

Nonionic surfactants which can be used advantageously are

1. alcohols,

2. alkanolamides, such as cocamides MEA/DEA/MIPA,

3. amine oxides, such as cocoamidopropylamine oxide,

4. esters which are formed by esterification of carboxylic acids with ethylene oxide, glycerol, sorbitan or other alcohols,

5. ethers, for example ethoxylated/propoxylated alcohols, ethoxylated/propoxylated esters, ethoxylated/propoxylated glycerol esters, ethoxylated/propoxylated cholesterols, ethoxylated/propoxylated triglyceride esters, ethoxylated/propoxylated lanolin, ethoxylated/propoxylated polysiloxanes, propoxylated POE ethers and alkyl polyglycosides, such as lauryl glucoside, decyl glycoside and cocoglycoside

6. sucrose esters, sucrose ethers

7. polyglycerol esters, diglycerol esters, monoglycerol esters

8. methyl glucose esters, esters of hydroxy acids

Also advantageous is the use of a combination of anionic and/or amphoteric surfactants with one or more nonionic surfactants.

The surface-active substance may be present in the preparations according to the invention in a concentration between 1 and 95% by weight, based on the total weight of the preparations.

The lipid phase of the cosmetic or dermatological emulsions according to the invention can advantageously be chosen from the following group of substances:

mineral oils, mineral waxes

oils, such as triglycerides of capric or of caprylic acid, and also natural oils such as, for example, castor oil;

fats, waxes and other natural and synthetic fatty substances, preferably esters of fatty acids with alcohols of low carbon number, e.g. with isopropanol, propylene glycol or glycerol, or esters of fatty alcohols with alkanoic acids of low carbon number or with fatty acids;

alkyl benzoates;

silicone oils, such as dimethylpolysiloxanes, diethylpolysiloxanes, diphenylpolysiloxanes and mixed forms thereof.

The oil phase of the emulsions of the present invention is advantageously chosen from the group of esters of saturated and/or unsaturated, branched and/or unbranched alkanecarboxylic acids having a chain length of from 3 to 30 carbon atoms and saturated and/or unsaturated, branched and/or unbranched alcohols having a chain length of from 3 to 30 carbon atoms, from the group of esters of aromatic carboxylic acids and saturated and/or unsaturated, branched and/or unbranched alcohols having a chain length of from 3 to 30 carbon atoms. Such ester oils can then advantageously be chosen from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate, and synthetic, semisynthetic and natural mixtures of such esters, e.g. jojoba oil.

In addition, the oil phase can advantageously be chosen from the group of branched and unbranched hydrocarbons and hydrocarbon waxes, of silicone oils, of dialkyl ethers, the group of saturated or unsaturated, branched or unbranched alcohols, and the fatty acid triglycerides, namely the triglycerol esters of saturated and/or unsaturated, branched and/or unbranched alkanecarboxylic acids having a chain length of from 8 to 24, in particular 12-18 carbon atoms. The fatty acid triglycerides can, for example, advantageously be chosen from the group of synthetic, semisynthetic and natural oils, e.g. olive oil, sunflower oil, soybean oil, groundnut oil, rapeseed oil, almond oil, palm oil, coconut oil, palm kernel oil and the like.

Any mixtures of such oil and wax components can also be used advantageously for the purposes of the present invention. It may also in some instances be advantageous to use waxes, for example cetyl palmitate, as the sole lipid component of the oil phase.

The oil phase is advantageously chosen from the group consisting of 2-ethylhexyl isostearate, octyldodecanol, isotridecyl isononanoate, isoeicosane, 2-ethylhexyl cocoate, C₁₂₋₁₅-alkyl benzoate, caprylic/capric triglyceride, dicaprylyl ether.

Particularly advantageous mixtures are those of C₁₂₋₁₅-alkyl benzoate and 2-ethylhexyl isostearate, mixtures of C₁₂₋₁₅-alkyl benzoate and isotridecyl isononanoate, and mixtures of C₁₂₋₁₅-alkyl benzoate, 2-ethylhexyl isostearate and isotridecyl isononanoate.

Of the hydrocarbons, paraffin oil, squalane and squalene are to be used advantageously for the purposes of the present invention.

The oil phase can advantageously also have a content of cyclic or linear silicone oils, or consist entirely of such oils, although it is preferable to use an additional content of other oil phase components apart from the silicone oil or the silicone oils. Such silicones or silicone oils may be in the form of monomers, which are generally characterized by structural elements, as follows:

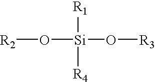

Linear silicones having two or more siloxyl units which are to be used advantageously according to the invention are generally characterized by structural elements, as follows:

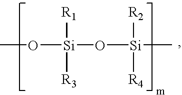

where the silicon atoms can be substituted by identical or different alkyl radicals and/or aryl radicals, which are shown here in general terms by the radicals R₁—R₄ (that is to say the number of different radicals is not necessarily limited to 4). m can assume values from 2-200 000.

Cyclic silicones to be used advantageously according to the invention are generally characterized by structural elements, as follows

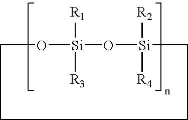

where the silicon atoms can be substituted by identical or different alkyl radicals and/or aryl radicals, which are shown here in general terms by the radicals R₁-R₄ (that is to say the number of different radicals is not necessarily limited to 4). n can assume values from 3/2 to 20. Fractions for n take into consideration that uneven numbers of siloxyl groups may be present in the cycle.

Advantageously, cyclomethicone (e.g. decamethylcyclopentasiloxane) is used as the silicone oil to be used according to the invention. However, other silicone oils are also to be used advantageously for the purpose of the present invention, for example undecamethylcyclotrisiloxane, polydimethylsiloxane, poly(methylphenylsiloxane), cetyldimethicone, behenoxydimethicone.

Also advantageous are mixtures of cyclomethicone and isotridecyl isononanoate, and those of cyclomethicone and 2-ethylhexyl isostearate.

It is, however, also advantageous to choose silicone oils of similar constitution to the above-described compounds whose organic side chains are derivatized, for example polyethoxylated and/or polypropoxylated. These include, for example, polysiloxanepolyalkyl-polyether copolymers, such as cetyl-dimethicone copolyol, (cetyl-dimethicone copolyol (and) polyglyceryl-4-isostearate (and) hexyl laurate).

Also particularly advantageous are mixtures of cyclomethicone and isotridecyl isononanoate, and of cyclomethicone and 2-ethylhexyl isostearate.

The aqueous phase of the preparations according to the invention optionally advantageously comprises alcohols, diols or polyols of low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, and also alcohols of low carbon number, e.g. ethanol, isopropanol, 1,2-propanediol, glycerol, and, in particular, one or more thickeners which can advantageously be chosen from the group consisting of silicon dioxide and aluminum silicates.

Preparations according to the invention in the form of emulsions advantageously comprise, in particular, one or more hydrocolloids. These hydrocolloids can advantageously be chosen from the group of gums, polysaccharides, cellulose derivatives, phyllosilicates, polyacrylates and/or other polymers.

Preparations according to the invention in the form of hydrogels comprise one or more hydrocolloids. These hydrocolloids can advantageously be chosen from the above-mentioned group.

The gums include saps from plants or trees which harden in the air and form resins, or extracts from aquatic plants. From this group, for the purposes of the present invention, gum arabic, carob flour, tragacanth, karaya, guar gum, pectin, gellan gum, carrageen, agar, algins, chondrus, xanthan gum, for example, can be chosen advantageously.

Also advantageous is the use of derivatized gums, such as, for example, hydroxypropyl guar (Jaguar®) HP 8).

The polysaccharides and polysaccharide derivatives include, for example, hyaluronic acid, chitin and chitosan, chondroitin sulfates, starch and starch derivatives.

The cellulose derivatives include, for example, methylcellulose, carboxymethylcellulose, hydroxyethylcellulose, hydroxypropylmethylcellulose.

The phyllosilicates include naturally occurring and synthetic clay earths, such as, for example, montmorillonite, bentonite, hectorite, laponite, magnesium aluminum silicates such as Veegum®. These can be used as such or in modified form, such as, for example, stearylalkonium hectorites.

In addition, silica gels can also be used advantageously.

The polyacrylates include, for example, Carbopol grades from Goodrich (Carbopol 980, 981, 1382, 5984, 2984, EDT 2001 or Pemulen TR2).

The polymers include, for example, polyacrylamides (Seppigel 305), polyvinyl alcohols, PVP, PVPNA copolymers, polyglycols.

Preparations according to the invention in the form of emulsions comprise one or more emulsifiers. These emulsifiers can advantageously be chosen from the group of nonionic, anionic, cationic or amphoteric emulsifiers.

The nonionic emulsifiers include

a) partial fatty acid esters and fatty acid esters of polyhydric alcohols and ethoxylated derivatives thereof (e.g. glyceryl monostearates, sorbitan stearates, glyceryl stearyl citrates, sucrose stearates)

b) ethoxylated fatty alcohols and fatty acids

c) ethoxylated fatty amines, fatty acid amides, fatty acid alkanolamides

d) alkylphenol polyglycol ethers (e.g. Triton X).

The anionic emulsifiers include

a) soaps (e.g. sodium stearate)

b) fatty alcohol sulfates

c) mono-, di- and trialkylphosphoric esters and ethoxylates thereof.

The cationic emulsifiers include

a) quaternary ammonium compounds with a long-chain aliphatic radical, e.g. distearyldimonium chloride.

The amphoteric emulsifiers include

a) alkylamininoalkanecarboxylic acids

b) betaines, sulfobetaines

c) imidazoline derivatives.

In addition, there are naturally occurring emulsifiers, which include beeswax, wool wax, lecithin and sterols.

O/W emulsifiers can be advantageously chosen, for example, from the group of polyethoxylated or polypropoxylated or polyethoxylated and polypropoxylated products, e.g.:

fatty alcohol ethoxylates,

ethoxylated wool wax alcohols,

polyethylene glycol ethers of the general formula R—O—(—CH₂—CH₂—O—)ₙ—R′,

fatty acid ethoxylates of the general formula R—COO—(—CH₂—CH₂—O—)ₙ—H,

etherified fatty acid ethoxylates of the general formula R—COO—(—CH₂—CH₂—O—)ₙ—R′,

esterified fatty acid ethoxylates of the general formula R—COO—(—CH₂—CH₂—O—)ₙ—C(O)—R′,

polyethylene glycol glycerol fatty acid esters,

ethoxylated sorbitan esters,

cholesterol ethoxylates,

ethoxylated triglycerides,

alkyl ether carboxylic acids of the general formula R—O—(—CH₂—CH₂—O—)ₙ—CH₂—COOH and n are a number from 5 to 30,

polyoxyethylene sorbitol fatty acid esters,

alkyl ether sulfates of the general formula R—O—(—CH₂—CH₂—O—)ₙ—SO₃—H,

fatty alcohol propoxylates of the general formula R—O—(—CH₂—CH(CH₃)—O—)ₙ—H,

polypropylene glycol ethers of the general formula R—O—(—CH₂—CH(CH₃)—O—)ₙ—R′,

propoxylated wool wax alcohols,

etherified fatty acid propoxylates R—COO—(—CH₂—CH(CH₃)—O—)ₙ—R′,

esterified fatty acid propoxylates of the general formula

R—COO—(—CH₂—CH(CH₃)—O—)ₙ—C(O)—R′,

fatty acid propoxylates of the general formula R—COO—(—CH₂—CH(CH₃)—O—)ₙ—H,

polypropylene glycol glycerol fatty acid esters,

propoxylated sorbitan esters,

cholesterol propoxylates,

propoxylated triglycerides,

alkyl ether carboxylic acids of the general formula R—O—(—CH₂—CH(CH₃)O—)ₙ—CH₂—COOH,

alkyl ether sulfates or the parent acids of these sulfates of the general formula R—O—(—CH₂—CH(CH₃)—O—)ₙ—SO₃—H,

fatty alcohol ethoxylates/propoxylates of the general formula R—O—Xₙ—Yₘ—H,

polypropylene glycol ethers of the general formula R—O—Xₙ—Yₘ—R′,

etherified fatty acid propoxylates of the general formula R—COO—Xₙ—Yₘ—R′,

fatty acid ethoxylates/propoxylates of the general formula R—COO—Xₙ—Yₘ—H.

According to the invention, particularly advantageous polyethoxylated or polypropoxylated or polyethoxylated and polypropoxylated O/W emulsifiers used are those chosen from the group of substances having HLB values of 11-18, very particularly advantageously having having HLB values of 14.5-15.5, provided the O/W emulsifiers have saturated radicals R and R′. If the O/W emulsifiers have unsaturated radicals R and/or R′, or isoalkyl derivatives are present, then the preferred HLB value of such emulsifiers can also be lower or higher.

It is advantageous to choose the fatty alcohol ethoxylates from the group of ethoxylated stearyl alcohols, cetyl alcohols, cetylstearyl alcohols (cetearyl alcohols). Particular preference is given to:

polyethylene glycol(13) stearyl ether (steareth-13), polyethylene glycol(14) stearyl ether (steareth-14), polyethylene glycol(15) stearyl ether (steareth-15), polyethylene glycol(16) stearyl ether (steareth-16), polyethylene glycol(17) stearyl ether (steareth-17), polyethylene glycol(18) stearyl ether (steareth-18), polyethylene glycol(19) stearyl ether (steareth-19), polyethylene glycol(20) stearyl ether (steareth-20),

polyethylene glycol(12) isostearyl ether (isosteareth-12), polyethylene glycol(13) isostearyl ether (isosteareth-13), polyethylene glycol(14) isostearyl ether (isosteareth-14),

polyethylene glycol(15) isostearyl ether (isosteareth-15), polyethylene glycol(16) isostearyl ether (isosteareth-16), polyethylene glycol(17) isostearyl ether (isosteareth-17),

polyethylene glycol(18) isostearyl ether (isosteareth-18), polyethylene glycol(19) isostearyl ether (isosteareth-19), polyethylene glycol(20) isostearyl ether (isosteareth-20),

polyethylene glycol(13) cetyl ether (ceteth-13), polyethylene glycol(14) cetyl ether (ceteth-14), polyethylene glycol(15) cetyl ether (ceteth-15), polyethylene glycol(16) cetyl ether (ceteth-16), polyethylene glycol(17) cetyl ether (ceteth-17), polyethylene glycol(18) cetyl ether (ceteth-18), polyethylene glycol(19) cetyl ether (ceteth-19), polyethylene glycol(20) cetyl ether (ceteth-20),

polyethylene glycol(13) isocetyl ether (isoceteth-13), polyethylene glycol(14) isocetyl ether (isoceteth-14), polyethylene glycol(15) isocetyl ether (isoceteth-15), polyethylene glycol(16) isocetyl ether (isoceteth-16), polyethylene glycol(17) isocetyl ether (isoceteth-17), polyethylene glycol(18) isocetyl ether (isoceteth-18), polyethylene glycol(19) isocetyl ether (isoceteth-19), polyethylene glycol(20) isocetyl ether (isoceteth-20),

polyethylene glycol(12) oleyl ether (oleth-12), polyethylene glycol(13) oleyl ether (oleth-13), polyethylene glycol(14) oleyl ether (oleth-14), polyethylene glycol(15) oleyl ether (oleth-15),

polyethylene glycol(12) lauryl ether (laureth-12), polyethylene glycol(12) isolauryl ether (isolaureth-12),

polyethylene glycol(13) cetylstearyl ether (ceteareth-13), polyethylene glycol(14) cetylstearyl ether (ceteareth-14), polyethylene glycol(15) cetylstearyl ether (ceteareth-15), polyethylene glycol(16) cetylstearyl ether (ceteareth-16), polyethylene glycol(17) cetylstearyl ether (ceteareth-17), polyethylene glycol(18) cetylstearyl ether (ceteareth-18), polyethylene glycol (19) cetylstearyl ether (ceteareth-19), polyethylene glycol(20) cetylstearyl ether (ceteareth-20).

It is also advantageous to choose the fatty acid ethoxylates from the following group:

polyethylene glycol(20) stearate, polyethylene glycol(21) stearate, polyethylene glycol(22) stearate, polyethylene glycol(23) stearate, polyethylene glycol(24) stearate, polyethylene glycol(25) stearate,

polyethylene glycol(12) isostearate, polyethylene glycol(13) isostearate, polyethylene glycol(14) isostearate, polyethylene glycol(15) isostearate, polyethylene glycol(16) isostearate, polyethylene glycol(17) isostearate, polyethylene glycol(18) isostearate, polyethylene glycol(19) isostearate, polyethylene glycol(20) isostearate, polyethylene glycol(21) isostearate, polyethylene glycol(22) isostearate, polyethylene glycol(23) isostearate, polyethylene glycol(24) isostearate, polyethylene glycol(25) isostearate, polyethylene glycol(12) oleate, polyethylene glycol(13) oleate, polyethylene glycol(14) oleate, polyethylene glycol(15) oleate, polyethylene glycol(16) oleate, polyethylene glycol(17) oleate, polyethylene glycol(18) oleate, polyethylene glycol(19) oleate, polyethylene glycol(20) oleate.

The ethoxylated alkyl ether carboxylic acid or salt thereof which can be used is advantageously sodium laureth-11 carboxylate.

Sodium laureth1-4 sulfate can be used advantageously as alkyl ether sulfate.

An advantageous ethoxylated cholesterol derivative which can be used is polyethylene glycol(30) cholesteryl ether. Polyethylene glycol(25) soyasterol has also proven successful.

Ethoxylated triglycerides which can be advantageously used are polyethylene glycol(60) Evening Primrose glycerides.

It is also advantageous to choose the polyethylene glycol glycerol fatty acid esters from the group polyethylene glycol(20) glyceryl laurate, polyethylene glycol(21) glyceryl laurate, polyethylene glycol(22) glyceryl laurate, polyethylene glycol(23) glyceryl laurate, polyethylene glycol(6) glyceryl caprate, polyethylene glycol(20) glyceryl oleate, polyethylene glycol(20) glyceryl isostearate, polyethylene glycol(18) glyceryl oleate/cocoate.

It is likewise favorable to choose the sorbitan esters from the group polyethylene glycol(20) sorbitan monolaurate, polyethylene glycol(20) sorbitan monostearate, polyethylene glycol(20) sorbitan monoisostearate, polyethylene glycol(20) sorbitan monopalmitate, polyethylene glycol(20) sorbitan monooleate.

Advantageous W/O emulsifiers which can be used are: fatty alcohols having 8 to 30 carbon atoms, monoglycerol esters of saturated and/or unsaturated, branched and/or unbranched alkanecarboxylic acids having a chain length of from 8 to 24, in particular 12-18, carbon atoms, diglycerol esters of saturated and/or unsaturated, branched and/or unbranched alkanecarboxylic acids having a chain length of from 8 to 24, in particular 12-18, carbon atoms, monoglycerol ethers of saturated and/or unsaturated, branched and/or unbranched alcohols having a chain length of from 8 to 24, in particular 12-18, carbon atoms, diglycerol ethers of saturated and/or unsaturated, branched and/or unbranched alcohols having a chain length of from 8 to 24, in particular 12-18, carbon atoms, propylene glycol esters of saturated and/or unsaturated, branched and/or unbranched alkanecarboxylic acids having a chain length of from 8 to 24, in particular 12-18, carbon atoms, and sorbitan esters of saturated and/or unsaturated, branched and/or unbranched alkanecarboxylic acids having a chain length of from 8 to 24, in particular 12-18, carbon atoms.

Particularly advantageous W/O emulsifiers are glyceryl monostearate, glyceryl monoisostearate, glyceryl monomyristate, glyceryl monooleate, diglyceryl monostearate, diglyceryl monoisostearate, propylene glycol monostearate, propylene glycol monoisostearate, propylene glycol monocaprylate, propylene glycol monolaurate, sorbitan monoisostearate, sorbitan monolaurate, sorbitan monocaprylate, sorbitan monoisooleate, sucrose distearate, cetyl alcohol, stearyl alcohol, arachidyl alcohol, behenyl alcohol, isobehenyl alcohol, selachyl alcohol, chimyl alcohol, polyethylene glycol(2) stearyl ether (steareth-2), glyceryl monolaurate, glyceryl monocaprate, glyceryl monocaprylate.

The examples below are intended to illustrate the invention, but not limit it. The numbers given refer to % by weight, unless stated otherwise.

### EXAMPLE 1

| PIT Spray | % by wt. |
|---|---|
| Glycerol monostearate SE | 0.50 |
| Polyoxyethylene(30) cetylstearyl ether | 5.00 |
| Cetyl alcohol | 2.50 |
| Dioctylbutamidotriazone | 1.00 |
| Ethylhexyltriazone | 4.00 |
| Phenylbenzimidazolesulfonic acid | 0.50 |
| Titanium dioxide | 0.50 |
| Zinc oxide | 2.00 |
| Butylene glycol dicaprylate/dicaprate | 5.00 |
| Phenyltrimethicone | 2.00 |
| PVP Hexadecene copolymer | 0.50 |
| Glycerol | 3.00 |
| Vitamin E acetate | 0.50 |
| Lipoic acid | 0.20 |
| Alpha-Glucosylrutin | 0.10 |
| DMDM Hydantoin | 0.60 |
| Phenoxyethanol | 0.50 |
| Ethanol | 3.00 |
| Perfume | q.s. |
| Water | ad 100.00 |

The constituents of the oil phase are combined and homogenized, then combined with the water phase and brought to a temperature of 80-85° C. (i.e. into the phase-inversion temperature range of the system), then cooled to room temperature (i.e. brought out of the phase-inversion temperature range of the system again).

### EXAMPLE 2

| PIT Spray | % by wt. |
|---|---|
| Polyoxyethylene(12) cetylstearyl ether | 5.00 |
| Cetyl alcohol | 1.00 |
| Anisotriazine | 1.50 |
| Dioctylbutamidotriazone | 2.00 |
| 4-Methylbenzylidenecamphor | 4.00 |
| Octocrylene | 4.00 |
| C₁₂₋₁₅ Alkyl benzoates | 2.50 |
| Titanium dioxide | 1.00 |
| Dimethicone | 0.50 |
| Shea Butter | 2.00 |
| Glycerol | 7.50 |
| Lipoic acid | 0.50 |
| Koncyl-L ® | 0.20 |
| Methylparaben | 0.50 |
| Phenoxyethanol | 0.40 |
| Ethanol | 2.00 |
| Perfume | q.s. |
| Water | ad 100.00 |

The constituents of the oil phase are combined and homogenized, then combined with the water phase and brought to a temperature of 80-85° C. (i.e. into the phase-inversion temperature range of the system), then cooled to room temperature (i.e. brought out of the phase-inversion temperature range of the system again).

### EXAMPLE 3

| PIT Spray | % by wt. |
|---|---|
| Glycerol monostearate SE | 3.00 |
| Polyoxyethylene(30) cetylstearyl ether | 1.00 |
| Stearyl alcohol | 3.00 |
| Butylmethoxydibenzoylmethane | 2.00 |
| Ethylhexyltriazone | 3.00 |
| Bisimidazylate | 0.50 |
| Zinc oxide | 3.00 |
| Dicaprylyl ether | 3.50 |
| Dicaprylyl carbonate | 6.00 |
| Dimethicone | 1.00 |
| Glycerol | 5.00 |
| Vitamin E acetate | 0.25 |
| Lipoic acid | 1.50 |
| Alpha-glucosylrutin | 0.20 |
| DMDM Hydantoin | 0.40 |
| Methylparaben | 0.25 |
| Ethanol | 1.50 |
| Perfume | q.s. |
| Water | ad 100.00 |

The constituents of the oil phase are combined and homogenized, then combined with the water phase and brought to a temperature of 80-85° C. (i.e. into the phase-inversion temperature range of the system), then cooled to room temperature (i.e. brought out of the phase-inversion temperature range of the system again).

### EXAMPLE 4

| PIT Spray | % by wt. |
|---|---|
| Glycerol monostearate SE | 2.00 |
| Polyoxyethylene(12) cetylstearyl ether | 1.00 |
| Polyoxyethylene(20) cetylstearyl ether | 2.00 |
| Cetyl alcohol | 1.50 |
| Ethylhexyl methoxycinnamate | 5.00 |
| Anisotriazine | 2.00 |
| Dioctylbutamidotriazone | 2.00 |
| Ethylhexyltriazone | 4.00 |
| Phenylbenzimidazole sulfonic acid | 3.00 |
| Titanium dioxide | 3.00 |
| Zinc oxide | 0.50 |
| Butylene glycol dicaprylate/dicaprate | 6.00 |
| Phenyltrimethicone | 0.50 |
| PVP Hexadecene copolymer | 0.50 |
| Glycerol | 7.50 |
| Lipoic acid | 0.50 |
| Alpha-glucosylrutin | 0.15 |
| DMDM hydantoin | 0.20 |
| Methylparaben | 0.15 |
| Phenoxyethanol | 1.00 |
| Perfume | q.s. |
| Water | ad 100.00 |

The constituents of the oil phase are combined and homogenized, then combined with the water phase and brought to a temperature of 80-85° C. (i.e. into the phase-inversion temperature range of the system), then cooled to room temperature (i.e. brought out of the phase-inversion temperature range of the system again).

### EXAMPLE 5

| PIT Spray | % by wt. |
|---|---|
| Glycerol monostearate SE | 4.00 |
| Polyoxyethylene(12) cetylstearyl ether | 1.50 |
| Stearyl alcohol | 0.50 |
| Ethylhexyl methoxycinnamate | 8.00 |
| Anisotriazine | 2.50 |
| 4-Methylbenzylidenecamphor | 2.00 |
| Octocrylene | 2.50 |
| Bisimidazylate | 1.50 |
| C₁₂₋₁₅ Alkyl benzoates | 5.00 |
| Titanium dioxide | 2.00 |
| Zinc oxide | 1.00 |
| Dicaprylyl carbonate | 2.00 |
| Phenyltrimethicone | 0.50 |
| Shea Butter | 0.50 |
| PVP Hexadecene copolymer | 1.00 |
| Glycerol | 2.50 |
| Vitamin E acetate | 1.00 |
| Lipoic acid | 0.40 |
| Koncyl-L ® | 0.15 |
| Phenoxyethanol | 0.60 |
| Ethanol | 1.00 |
| Perfume | q.s. |
| Water | ad 100.00 |

The constituents of the oil phase are combined and homogenized, then combined with the water phase and brought to a temperature of 80-85° C. (i.e. into the phase-inversion temperature range of the system), then cooled to room temperature (i.e. brought out of the phase-inversion temperature range of the system again).

### EXAMPLE 6

| O/W Emulsion | % by wt. |
|---|---|
| Glycerol monostearate SE | 0.50 |
| Glyceryl stearate citrate | 2.00 |
| PEG-40 stearate | 0.50 |
| Cetyl alcohol | 2.50 |
| Butylmethoxydibenzoylmethane | 1.00 |
| Ethylhexyltriazone | 4.00 |
| 4-Methylbenzylidenecamphor | 4.00 |
| Dioctylbutamidotriazone | 1.00 |
| Bisimidazylate | 1.00 |
| Phenylbenzimidazolesulfonic acid | 0.50 |
| Titanium dioxide | 1.00 |
| Zinc oxide | 2.00 |
| Butylene glycol dicaprylate/dicaprate | 5.00 |
| Cyclomethicone | 2.00 |
| PVP Hexadecene copolymer | 0.50 |
| Glycerol | 3.00 |
| Xanthan gum | 0.15 |
| Vitamin E acetate | 0.50 |
| Lipoic acid | 0.50 |
| Methylparaben | 0.15 |
| Phenoxyethanol | 1.00 |
| Perfume | q.s. |
| Water | ad 100.00 |

The constituents of the oil and water phase in each case are combined, the two phases are combined at 70-75° C. and homogenized and then cooled to room temperature.

### EXAMPLE 7

| O/W Emulsion | % by wt. |
|---|---|
| Glycerol monostearate SE | 1.00 |
| Stearic acid | 3.00 |
| Cetyl alcohol | 1.00 |
| Anisotriazine | 1.50 |
| Dioctylbutamidotriazone | 2.00 |
| 4-Methylbenzylidenecamphor | 4.00 |
| Octocrylene | 4.00 |
| Titanium dioxide | 1.50 |
| Zinc oxide | 1.00 |
| C₁₂₋₁₅ Alkyl benzoates | 2.50 |
| Dimethicone | 0.50 |
| Shea Butter | 2.00 |
| Glycerol | 7.50 |
| Sodium carbomer | 0.20 |
| Lipoic acid | 0.20 |
| DMDM Hydantoin | 0.60 |
| Phenoxyethanol | 0.40 |
| Ethanol | 2.00 |
| Perfume | q.s. |
| Water | ad 100.00 |

The constituents of the oil and water phase in each case are combined, the two phases are combined at 70-75° C. and homogenized and then cooled to room temperature.

### EXAMPLE 8

| O/W Emulsion | % by wt. |
|---|---|
| Glycerol monostearate SE | 3.00 |
| Stearyl alcohol | 3.00 |
| Butylmethoxydibenzoylmethane | 2.00 |
| Ethylhexyltriazone | 3.00 |
| Bisimidazylate | 0.50 |
| Dicaprylyl ether | 3.50 |
| Dicaprylyl carbonate | 6.00 |
| Dimethicone | 1.00 |
| Xanthan gum | 0.05 |
| Sodium carbomer | 0.10 |
| Vitamin E acetate | 0.25 |
| Lipoic acid | 1.50 |
| DMDM hydantoin | 0.40 |
| Methylparaben | 0.25 |
| Ethanol | 1.50 |
| Perfume | q.s. |
| Water | ad 100.00 |

The constituents of the oil and water phase in each case are combined, the two phases are combined at 70-75° C. and homogenized and then cooled to room temperature.

### EXAMPLE 9

| O/W Emulsion | % by wt. |
|---|---|
| Glyceryl stearate citrate | 1.00 |
| Stearic acid | 2.00 |
| Cetyl alcohol | 1.50 |
| Ethylhexyl methoxycinnamate | 5.00 |
| Anisotriazine | 2.00 |
| Dioctylbutamidotriazone | 2.00 |
| Ethylhexyltriazone | 4.00 |
| Dioctylbutamidotriazone | 2.00 |
| Phenylbenzimidazolesulfonic acid | 3.00 |
| Titanium dioxide | 3.00 |
| Zinc oxide | 0.50 |
| Butylene glycol dicaprylate/dicaprate | 6.00 |
| Cyclomethicone | 0.50 |
| PVP hexadecene copolymer | 0.50 |
| Glycerol | 7.50 |
| Sodium carbomer | 0.20 |
| Lipoic acid | 0.50 |
| Biotin | 0.04 |
| DMDM hydantoin | 0.20 |
| Koncyl-L ® | 0.18 |
| Phenoxyethanol | 0.40 |
| Perfume | q.s. |
| Water | ad 100.00 |

The constituents of the oil and water phase in each case are combined, the two phases are combined at 70-75° C. and homogenized and then cooled to room temperature.

### EXAMPLE 10

| O/W Emulsion | % by wt. |
|---|---|
| Glyceryl stearate citrate | 2.00 |
| Cetyl phosphate | 1.00 |
| Cetyl alcohol | 0.50 |
| Ethylhexyl methoxycinnamate | 6.00 |
| Anisotriazine | 2.50 |
| Ethylhexyltriazone | 4.00 |
| 4-Methylbenzylidenecamphor | 2.00 |
| Dioctylbutamidotriazone | 1.00 |
| Titanium dioxide | 2.00 |
| C₁₂₋₁₅ Alkyl benzoates | 4.00 |
| Dicaprylyl ether | 2.00 |
| Dimethicone | 2.00 |
| PVP hexadecene copolymer | 1.00 |
| Glycerol | 5.00 |
| Vitamin E acetate | 0.75 |
| Lipoic acid | 0.60 |
| Koncyl-L ® | 0.20 |
| Methylparaben | 0.50 |
| Phenoxyethanol | 0.50 |
| Ethanol | 3.00 |
| Perfume | q.s. |
| Water | ad 100.00 |

The constituents of the oil and water phase in each case are combined, the two phases are combined at 70-75° C. and homogenized and then cooled to room temperature.

### EXAMPLE 11

Example 11

| O/W Emulsion | % by wt. |
|---|---|
| Glycerol monostearate SE | 1.50 |
| PEG-40 stearate | 2.00 |
| Stearyl alcohol | 2.00 |
| Butylmethoxydibenzoylmethane | 2.00 |
| Dioctylbutamidotriazone | 2.00 |
| Ethylhexyltriazone | 2.00 |
| 4-Methylbenzylidenecamphor | 4.00 |
| Bisimidazylate | 1.00 |
| Titanium dioxide | 2.00 |
| Zinc oxide | 3.00 |
| C₁₂₋₁₅ Alkyl benzoates | 7.00 |
| Dicaprylyl carbonate | 2.00 |
| Lipoic acid | 1.00 |
| Biotin | 0.10 |
| Koncyl-L ® | 0.10 |
| Phenoxyethanol | 0.40 |
| Perfume | q.s. |
| Water | ad 100.00 |

The constituents of the oil and water phase in each case are combined, the two phases are combined at 70-75° C. and homogenized and then cooled to room temperature.

### EXAMPLE 12

| O/W Emulsion | % by wt. |
|---|---|
| Glyceryl stearate citrate | 2.50 |
| Stearyl alcohol | 0.50 |
| Cetyl alcohol | 2.00 |
| Ethylhexyl methoxycinnamate | 8.00 |
| Anisotriazine | 2.50 |
| 4-Methylbenzylidenecamphor | 2.00 |
| Octocrylene | 2.50 |
| Bisimidazylate | 1.50 |
| Zinc oxide | 5.00 |
| C₁₂₋₁₅ Alkyl benzoates | 5.00 |
| Dicaprylyl carbonate | 2.00 |
| Cyclomethicone | 0.50 |
| Shea Butter | 0.50 |
| PVP Hexadecene copolymer | 1.00 |
| Glycerol | 2.50 |
| Xanthan gum | 0.30 |
| Vitamin E acetate | 1.00 |
| Lipoic acid | 0.40 |
| Koncyl-L ® | 0.15 |
| Phenoxyethanol | 0.60 |
| Ethanol | 1.00 |
| Perfume | q.s. |
| Water | ad 100.00 |

The constituents of the oil and water phase in each case are combined, the two phases are combined at 70-75° C. and homogenized and then cooled to room temperature.

### EXAMPLE 13

| O/W Emulsion | % by wt. |
|---|---|
| Glyceryl stearate citrate | 2.00 |
| Cetyl stearyl alcohol | 1.00 |
| Octyldodecanol | 2.00 |
| Butylmethoxydibenzoylmethane | 2.00 |
| Dioctylbutamidotriazone | 2.00 |
| Ethylhexyltriazone | 2.00 |
| 4-Methylbenzylidenecamphor | 4.00 |
| Bisimidazylate | 1.00 |
| Titanium dioxide | 2.00 |
| Zinc oxide | 3.00 |
| Cyclomethicone | 3.00 |
| Glycerol | 3.00 |
| Sodium carbomer | 0.10 |
| Na₃HEDTA | 0.20 |
| DMDM Hydantoin | 0.20 |
| Koncyl-L ® | q.s. |
| Methylparaben | q.s. |
| Phenoxyethanol | q.s. |
| Perfume | q.s. |
| Sodium hydroxide solution | q.s. |
| Water | ad 100.00 |

The constituents of the oil and water phase in each case are combined, the two phases are combined at 70-75° C. and homogenized and then cooled to room temperature.

### EXAMPLE 14

| O/W Emulsion | % by wt. |
|---|---|
| Stearic acid | 2.50 |
| Cetyl alcohol | 3.00 |
| Octyldodecanol | 4.00 |
| Butylmethoxydibenzoylmethane | 0.50 |
| 4-Methylbenzylidenecamphor | 1.00 |
| Cyclomethicone | 0.50 |
| Glycerol | 5.00 |
| Sodium carbomer | 0.05 |
| Lipoic acid | 0.20 |
| Trisodium EDTA | 0.20 |
| Koncyl-L ® | q.s. |
| Methylparaben | q.s. |
| Phenoxyethanol | q.s. |
| Ethanol | 3.00 |
| Perfume | q.s. |
| Sodium hydroxide solution | 0.30 |
| Water | ad 100.00 |

The constituents of the oil and water phase in each case are combined, the two phases are combined at 70-75° C. and homogenized and then cooled to room temperature.

### EXAMPLE 15

| Hydrodispersion | % by wt. |
|---|---|
| Polyoxyethylene(20) cetylstearyl ether | 1.00 |
| Acrylates/C10-30 alkyl acrylate crosspolymer | 0.50 |
| Butylmethoxydibenzoylmethane | 1.00 |
| Ethylhexyltriazone | 4.00 |
| 4-Methylbenzylidenecamphor | 4.00 |
| Dioctylbutamidotriazone | 1.00 |
| Bisimidazylate | 1.00 |
| Phenylbenzmidazolesulfonic acid | 0.50 |
| Titanium dioxide | 0.50 |
| Zinc oxide | 0.50 |
| C₁₂₋₁₅ Alkyl benzoates | 2.00 |
| Butylene glycol dicaprylate/dicaprate | 4.00 |
| Phenyltrimethicone | 2.00 |
| PVP Hexadecene copolymer | 0.50 |
| Glycerol | 3.00 |
| Vitamin E acetate | 0.50 |
| Lipoic acid | 0.15 |
| Koncyl-L ® | 0.20 |
| Methylparaben | 0.50 |
| Phenoxyethanol | 0.50 |
| Ethanol | 3.00 |
| Perfume | q.s. |
| Water | ad 100.00 |

The constituents of the oil and water phase in each case are combined, the two phases are combined at 70-75° C. and homogenized and then cooled to room temperature.

### EXAMPLE 16

| Hydrodispersion | % by wt. |
|---|---|
| Sodium carbomer | 0.20 |
| Xanthan gum | 0.30 |
| Anisotriazine | 1.50 |
| Dioctylbutamidotriazone | 2.00 |
| 4-Methylbenzylidenecamphor | 4.00 |
| Octocrylene | 4.00 |
| Zinc oxide | 1.00 |
| C₁₂₋₁₅ Alkyl benzoates | 2.50 |
| Dicaprylyl ether | 4.00 |
| Dicaprylyl carbonate | 2.00 |
| Dimethicone | 0.50 |
| Shea Butter | 2.00 |
| Glycerol | 7.50 |
| Lipoic acid | 0.60 |
| DMDM Hydantoin | 0.60 |
| Phenoxyethanol | 0.40 |
| Ethanol | 2.00 |
| Perfume | q.s. |
| Water | ad 100.00 |

The constituents of the oil and water phase in each case are combined, the two phases are combined at 70-75° C. and homogenized and then cooled to room temperature.

### EXAMPLE 17

| Hydrodispersion | % by wt. |
|---|---|
| Cetyl alcohol | 1.00 |
| Acrylate/C₁₀₋₃₀ alkyl acrylate crosspolymer | 0.40 |
| Xanthan gum | 0.15 |
| Butylmethoxydibenzoylmethane | 2.00 |
| Ethylhexyltriazone | 3.00 |
| Octocrylene | 4.00 |
| Bisimidazylate | 0.50 |
| Titanium dioxide | 2.00 |
| Zinc oxide | 3.00 |
| Butylene glycol dicaprylate/dicaprate | 2.00 |
| Dicaprylyl carbonate | 6.00 |
| Dimethicone | 1.00 |
| Octoxyglycerol | 1.00 |
| Glycine soya | 1.50 |
| Vitamin E acetate | 0.25 |
| Lipoic acid | 1.50 |
| DMDM Hydantoin | 0.40 |
| Methylparaben | 0.25 |
| Ethanol | 1.50 |
| Perfume | q.s. |
| Water | ad 100.00 |

The constituents of the oil and water phase in each case are combined, the two phases are combined at 70-75° C. and homogenized and then cooled to room temperature.

### EXAMPLE 18

| Hydrodispersion | % by wt. |
|---|---|
| Polyoxyethylene(20) cetylstearyl ether | 0.5 |
| Sodium carbomer | 0.30 |
| Acrylate/C₁₀₋₃₀ alkyl acrylate crosspolymer | 0.10 |
| Ethylhexylmethoxy cinnamate | 5.00 |
| Anisotriazine | 2.00 |
| Dioctylbutamidotriazone | 2.00 |
| Ethylhexyltriazone | 4.00 |
| Dioctylbutamidotriazone | 2.00 |
| Phenylbenzimidazolesulfonic acid | 3.00 |
| Titanium dioxide | 3.00 |
| Butylene glycol dicaprylate/dicaprate | 6.00 |
| Phenyltrimethicone | 0.50 |
| PVP Hexadecene copolymer | 0.50 |
| Glycerol | 7.50 |
| Lipoic acid | 1.00 |
| DMDM Hydantoin | 0.20 |
| Methylparaben | 0.15 |
| Phenoxyethanol | 1.00 |
| Perfume | q.s. |
| Water | ad 100.00 |

The constituents of the oil and water phase in each case are combined, the two phases are combined at 70-75° C. and homogenized and then cooled to room temperature.

### EXAMPLE 19

| Hydrodispersion | % by wt. |
|---|---|
| Acrylate/C₁₀₋₃₀ alkyl acrylate crosspolymer | 0.10 |
| Xanthan gum | 0.50 |
| Ethylhexyl methoxycinnamate | 8.00 |
| Anisotriazine | 2.50 |
| Dioctylbutamidotriazone | 1.00 |
| 4-Methylbenzylidenecamphor | 2.00 |
| Octocrylene | 2.50 |
| Bisimidazylate | 2.00 |
| Titanium dioxide | 1.00 |
| Zinc oxide | 2.00 |
| Phenyltrimethicone | 2.00 |
| PVP Hexadecene copolymer | 1.00 |
| Octoxyglycerol | 0.50 |
| Glycerol | 2.50 |
| Vitamin E acetate | 1.00 |
| Lipoic acid | 0.80 |
| Koncyl-L ® | 0.15 |
| Phenoxyethanol | 0.60 |
| Ethanol | 1.00 |
| Perfume | q.s. |
| Water | ad 100.00 |

The constituents of the oil and water phase in each case are combined, the two phases are combined at 70-75° C. and homogenized and then cooled to room temperature.

### EXAMPLE 20

| W/O Emulsion | % by wt. |
|---|---|
| Polyglyceryl-2 dipolyhydroxystearate | 5.00 |
| Anisotriazine | 2.00 |
| Dioctylbutamidotriazone | 3.00 |
| Octocrylene | 7.00 |
| Dioctylbutamidotriazone | 1.00 |
| Bisimidazylate | 1.00 |
| Phenylbenzimidazolesulfonic acid | 0.50 |
| Zinc oxide | 3.00 |
| Dicaprylyl ether | 10.00 |
| Dicaprylyl carbonate | 5.00 |
| Cyclomethicone | 2.00 |
| PVP Hexadecene copolymer | 0.50 |
| Glycerol | 3.00 |
| MgSO₄ | 1.00 |
| Vitamin E acetate | 0.50 |
| Lipoic acid | 0.10 |
| Methylparaben | 0.50 |
| Phenoxyethanol | 0.50 |
| Ethanol | 3.00 |
| Perfume | q.s. |
| Water | ad 100.00 |

The constituents of the oil and water phase in each case are combined, the two phases are combined at 70-75° C. and homogenized and then cooled to room temperature.

### EXAMPLE 21

| W/O Emulsion | % by wt. |
|---|---|
| Cetyldimethicone copolyol | 2.50 |
| Ethylhexyl methoxycinnamate | 8.00 |
| Anisotriazine | 2.50 |
| Dioctylbutamidotriazone | 1.00 |
| 4-Methylbenzylidenecamphor | 2.00 |
| Octocrylene | 2.50 |
| Bisimidazylate | 2.00 |
| Titanium dioxide | 2.00 |
| Zinc oxide | 1.00 |
| Dimethicone | 4.00 |
| Cyclomethicone | 25.00 |
| Octoxyglycerol | 0.30 |
| Glycerol | 7.50 |
| Glycine Soya | 1.00 |
| MgSO₄ | 0.50 |
| Lipoic acid | 0.60 |
| DMDM Hydantoin | 0.60 |
| Phenoxyethanol | 0.40 |
| Perfume | q.s. |
| Water | ad 100.00 |

The constituents of the oil and water phase in each case are combined, the two phases are combined at 70-75° C. and homogenized and then cooled to room temperature.

### EXAMPLE 22

| W/O Emulsion | % by wt. |
|---|---|
| PEG-30 dipolyhydroxystearate | 5.00 |
| Butylmethoxydibenzoylmethane | 2.00 |
| Ethylhexyltriazone | 3.00 |
| Octocrylene | 4.00 |
| Bisimidazylate | 0.50 |
| Titanium dioxide | 1.50 |
| Zinc oxide | 2.00 |
| Mineral oil | 10.0 |
| Butylene glycol dicaprylate/dicaprate | 2.00 |
| Dicaprylyl carbonate | 6.00 |
| Dimethicone | 1.00 |
| Shea Butter | 3.00 |
| Octoxyglycerol | 1.00 |
| Glycine soya | 1.50 |
| MgCl₂ | 1.00 |
| Vitamin E acetate | 0.25 |
| Lipoic acid | 1.50 |
| DMDM Hydantoin | 0.40 |
| Methylparaben | 0.25 |
| Ethanol | 1.50 |
| Perfume | q.s. |
| Water | ad 100.00 |

The constituents of the oil and water phase in each case are combined, the two phases are combined at 70-75° C. and homogenized and then cooled to room temperature.

### EXAMPLE 23

| W/O Emulsion | % by wt. |
|---|---|
| Cetyldimethicone copolyol | 4.00 |
| Ethylhexyl methoxycinnamate | 5.00 |
| Anisotriazine | 2.00 |
| Butylmethoxydibenzoylmethane | 1.00 |
| Ethylhexyltriazone | 4.00 |
| 4-Methylbenzylidenecamphor | 4.00 |
| Dioctylbutamidotriazone | 2.00 |
| Phenylbenzimidazolesulfonic acid | 3.00 |
| Zinc oxide | 0.50 |
| C₁₂₋₁₅ Alkyl benzoates | 9.00 |
| Butylene glycol dicaprylate/dicaprate | 8.00 |
| Dimethicone | 5.00 |
| PVP Hexadecene copolymer | 0.50 |
| Glycerol | 7.50 |
| MgSO₄ | 0.50 |
| Lipoic acid | 1.00 |
| DMDM Hydantoin | 0.20 |
| Methylparaben | 0.15 |
| Phenoxyethanol | 1.00 |
| Perfume | q.s. |
| Water | ad 100.00 |

The constituents of the oil and water phase in each case are combined, the two phases are combined at 70-75° C. and homogenized and then cooled to room temperature.

### EXAMPLE 24

| W/O Emulsion | % by wt. |
|---|---|
| Polyglyceryl-2 dipolyhydroxystearate | 4.50 |
| Ethylhexyl methoxycinnamate | 4.00 |
| Anisotriazine | 2.50 |
| Dioctylbutamidotriazone | 3.00 |
| 4-Methylbenzylidenecamphor | 2.00 |
| Octocrylene | 2.50 |
| Phenylbenzimidazolesulfonic acid | 2.00 |
| Titanium dioxide | 3.00 |
| Mineral oil | 8.00 |
| Dicaprylyl ether | 7.00 |
| Butylene glycol dicaprylate/dicaprate | 4.00 |
| Cyclomethicone | 2.00 |
| PVP Hexadecene copolymer | 1.00 |
| Octoxyglycerol | 0.50 |
| Glycerol | 2.50 |
| MgCl₂ | 0.70 |
| Vitamin E acetate | 1.00 |
| Lipoic acid | 0.80 |
| Phenoxyethanol | 0.60 |
| Ethanol | 1.00 |
| Perfume | q.s. |
| Water | ad 100.00 |

The constituents of the oil and water phase in each case are combined, the two phases are combined at 70-75° C. and homogenized and then cooled to room temperature.

### EXAMPLE 25

| W/O Emulsion | % by wt. |
|---|---|
| Polyglyceryl-2 dipolyhydroxystearate | 4.00 |
| Wool wax alcohol | 0.50 |
| Isohexadecane | 1.00 |
| Myristyl myristate | 0.50 |
| Cera microcristallina + paraffinum liquidum | 1.00 |
| Butylmethoxydibenzoylmethane | 0.50 |
| 4-Methylbenzylidenecamphor | 1.00 |
| Butylene glycol dicaprylate/dicaprate | 4.00 |
| Glycerol | 5.00 |
| Vitamin E acetate | 0.50 |
| Lipoic acid | 0.20 |
| Na₃HEDTA | 0.20 |
| Methylparaben | q.s. |
| Phenoxyethanol | q.s. |
| Perfume | q.s. |
| Water | ad 100.00 |

The constituents of the oil and water phase in each case are combined, the two phases are combined at 70-75° C. and homogenized and then cooled to room temperature.

### EXAMPLE 26

| W/O Emulsion | % by wt. |
|---|---|
| Polyglyceryl-2 dipolyhydroxystearate | 5.00 |
| Wool wax alcohol | 1.50 |
| Isohexadecane | 2.00 |
| Myristyl myristate | 1.50 |
| Cera microcristallina + paraffinum liquidum | 2.00 |
| Butylmethoxydibenzoylmethane | 1.50 |
| 4-Methylbenzylidenecamphor | 3.00 |
| Butylene glycol dicaprylate/dicaprate | 5.00 |
| Shea Butter | 0.50 |
| Butylene glycol | 6.00 |
| Octoxyglycerol | 3.00 |
| Vitamin E acetate | 1.00 |
| Lipoic acid | 0.25 |
| Na₃HEDTA | 0.20 |
| Methylparaben | q.s. |
| Phenoxyethanol | q.s. |
| Ethanol | 3.00 |
| Perfume | q.s. |
| Water | ad 100.00 |

The constituents of the oil and water phase in each case are combined, the two phases are combined at 70-75° C. and homogenized and then cooled to room temperature.

1. The use of active ingredient combinations of
(a) α-lipoic acid and
(b) one or more dermatologically compatible substances which bring about light absorption in the UV-A region and/or UV-B region,
for the preparation of cosmetic or dermatological preparations for the preparation of cosmetic or dermatological preparations for the treatment and/or prophylaxis of pigmentation disorders.

2. The use as claimed in claim 1, characterized in that they comprise 0.001-10% by weight of α-lipoic acid, based on the total weight of the preparations.

3. The use as claimed in claim 1, characterized in that they comprise 0.001-10% by weight of one or more substances chosen from the group of dermatologically compatible substances which exhibit light absorption in the UV-A region and/or UV-B region.
